(19) United States
(12) Patent Application Publication
(10) Pub. No.: US 2026/0107893 A1
(43) Pub. Date: Apr. 23, 2026

(54) METHOD FOR EVALUATING INFLUENCE BY GENE-EDITED LOW-GOSSYPOL COTTON ON APHIS GOSSYPII

(71) Applicant: INSTITUTE OF COTTON RESEARCH OF CAAS, Anyang (CN)

(72) Inventors: Junyu LUO, Anyang (CN); Jinjie CUI, Anyang (CN); Xiangzhen ZHU, Anyang (CN); Kaixin ZHANG, Anyang (CN); Xueke GAO, Anyang (CN); Li WANG, Anyang (CN); Jichao JI, Anyang (CN); Dongyang LI, Anyang (CN)

(73) Assignee: INSTITUTE OF COTTON RESEARCH OF CAAS, Anyang (CN)

(21) Appl. No.: 18/918,747

(22) Filed: Oct. 17, 2024

(51) Int. Cl. A01H 1/00; A01K 67/033

(52) U.S. Cl. CPC A01H 1/127; A01K 67/30

(57) ABSTRACT

A method for evaluating an influence by gene-edited low-gossypol cotton on Aphis gossypii is provided, relating to the technical field of cotton safety evaluation. The method for evaluating an influence by gene-edited low-gossypol cotton on Aphis gossypii includes planting the gene-edited low-gossypol cotton indoors, collecting young leaves and inoculating nymphal Aphis gossypii on the leaves, rearing the nymphal Aphis gossypii, recording a number of adult Aphis gossypii deaths and a number of newly hatched nymphal Aphis gossypii, calculating an Aphis gossypii mortality rate and an average Aphis gossypii productivity, and evaluating the influence.

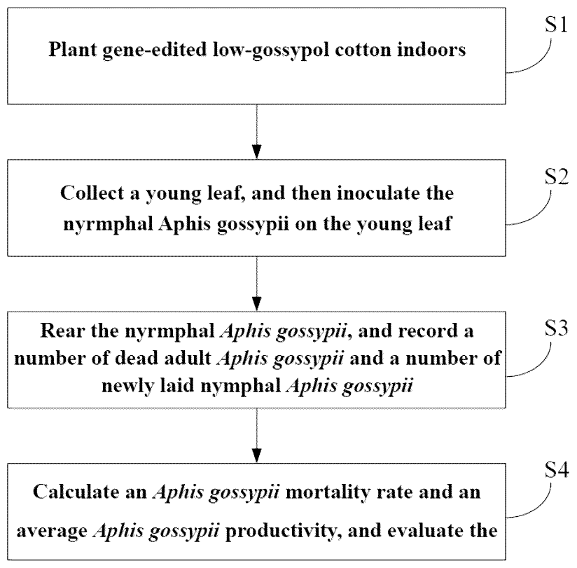

### TECHNICAL FIELD

The present disclosure relates to the technical field of influence evaluation by cotton, and in particular to a method for evaluating an influence by gene-edited low-gossypol cotton on Aphis gossypii.

### BACKGROUND

Cotton is one of the most important economic crops in the world, and cotton fiber is the main raw material for the textile industry. However, common cotton varieties have problems with pests and diseases, poor environmental adaptability, low resource utilization efficiency, unstable fiber quality, and high production costs. Therefore, it has become a new direction for the industry's development by breeding novel cotton varieties to overcome the above problems.

The development of gene editing technology provides new ideas for cotton breeding. Through the gene editing technology, a cotton genome can be precisely modified to improve its specific properties. Gene-edited low-gossypol cotton is a specific application of the gene editing technology in cotton breeding. Gossypol is a secondary metabolite naturally occurring in cotton that is toxic and harmful to human beings and many animals. In traditional cotton varieties, the gossypol is mainly present in cotton seeds and other plant tissues, and limits the consumption and utilization of cotton seeds. Through the gene editing technology, such as CRISPR-Cas9, key genes in a gossypol biosynthesis pathway of the cotton can be precisely knocked out or regulated, thereby significantly reducing the gossypol content. This gene-edited low-gossypol cotton not only retains the excellent characteristics of the original cotton variety, but also makes cottonseed oil and cottonseed protein safe for consumption, thereby increasing an economic value of cotton and expanding an application range.

Many materials from gene-edited low-gossypol cotton have been approved for productive experiments, but their influence still remains unclear on the cotton field ecosystem. Gene-edited cotton may have influences on non-target organisms (such as natural enemies and pollinators), change the population dynamics of pests, and affect ecosystem service functions such as biodiversity and soil microorganisms. Therefore, accurately evaluating the influence by gene-edited cotton on the cotton field ecosystem is of important implications for ensuring ecological security, protecting biodiversity and other issues. Systematic assessment of these influences can identify and manage potential ecological risks in advance, thus ensuring that long-term cultivation by gene-edited cotton does not have negative influences on the ecosystem. These evaluation results also provide a scientific basis for policymakers and regulators to ensure the safe use and promotion of gene-edited crops.

Aphis gossypii plays an important role in the cotton field ecosystem. The Aphis gossypii sucks the sap from cotton leaves and tender cotton tips, causing the cotton leaves to curl and the plants to stunt, and secretes honeydew, affecting photosynthesis and the normal growth of cotton. As a result, the Aphis gossypii is regarded as a major pest in the cotton field ecosystem. In addition, aphids have complex ecological relationships with a variety of predatory natural enemies (such as ladybugs and lacewings), which play a key role in controlling the number of aphids, thereby forming a dynamically balanced ecosystem. Aphids are extremely sensitive to environmental conditions (such as temperature, humidity, and plant nutritional status). Environmental changes can directly affect the reproduction and spread of aphids, thereby affecting the entire cotton field ecosystem. Moreover, aphids occupy an important nutritional level in the food chain of cotton field ecosystems. Changes in a number and behavior of the aphids may affect higher nutritional levels, including predatory natural enemies, parasitic natural enemies and other organisms. By studying the group response of aphids, it is able to indirectly understand the influence by gene-edited low-gossypol cotton on the cotton field food chain, food web, and even the entire ecosystem.

Therefore, it is of great significance to improving cotton yield and quality to provide an evaluation method for the influence by gene-edited low-gossypol cotton on Aphis gossypii with a short experimental cycle, rapid evaluation conclusions, controllable experimental conditions, reduced interference from environmental factors, and accurate and reliable data. This is of great significance to ensuring the sustainable development of the whole cotton industry.

### SUMMARY

To solve the above problems, the present disclosure provides a method for evaluating an influence by gene-edited low-gossypol cotton on Aphis gossypii, including the following steps:
S1, planting the gene-edited low-gossypol cotton indoors;
S2, collecting a fully expanded young leaf from a top part of a seedling of the cotton, placing the young leaf into a medium contained in a container, and then inoculating nymphal Aphis gossypii on the young leaf;
S3, rearing the nymphal Aphis gossypii until the 6th day after it is developed into an adult Aphis gossypii and lay new nymphal Aphis gossypii, recording a number of dead adult Aphis gossypii and a number of newly laid nymphal Aphis gossypii every 24 h, and terminating investigation when all Aphis gossypii individuals die in an initial test; and
S4, calculating an Aphis gossypii mortality rate and an average Aphis gossypii productivity, and evaluating the influence of the gene-edited low-gossypol cotton on the Aphis gossypii based on the Aphis gossypii mortality rate and the average Aphis gossypii productivity.

Further, the young leaves are collected from seedlings of the cotton with consistent growth after a cotton seedling has 2 to 4 fully expanded leaves.

Further, the medium is an agar-containing medium.

Further, the medium has an agar concentration of 5 wt ‰ to 10 wt ‰.

Further, 5 to 8 wingless Aphis gossypii individuals are inoculated on the young leaf of the cotton in each container, and the adult Aphis gossypii is removed while only 5 initially hatched nymphal Aphis gossypii individuals are retained as Aphis gossypii of the initial test after 24 h.

Further, the rearing is conducted at 24° C.±1° C. under a humidity of 50%±10%.

Further, the rearing is conducted at a photoperiod of L (light):D (darkness)=16 h: 8 h.

Further, a petiole is kept intact and then inserted into the medium while a young leave is kept completely placed on the agar-containing medium during the rearing.

Further, a fresh fully expanded young leaf from the top part of a same material for test (i.e., cotton) is replaced every 2 d to 3 d during the rearing to ensure an adequate nutritional intake of the Aphis gossypii individuals in the test.

Further, newly hatched nymphal Aphis gossypii individuals are removed every day during the rearing.

Further, a calculation formula of the Aphis gossypii mortality rate is as follows:

M=n/N×100

In the above formula, M represents the Aphis gossypii mortality rate, in percentage (%); n represents a number of dead aphids on the 6th day after the test is started; and N represents a number of aphids inoculated.

Further, a calculation formula of the average Aphis gossypii productivity is as follows:

$$X = \frac{{\sum }_{i = 1}^{10} {X}_{i}}{50}$$

In the formula, X represents an average Aphis gossypii productivity; i represents a total number of containers; Xᵢ represents a total number of nymphal Aphis gossypii produced by initial Aphis gossypii in an i-th container; and 50 represents a total number of the initial Aphis gossypii in 10 containers.

When X≤10, it represents that the low-gossypol cotton is highly resistant to the Aphis gossypii (high resistance); when 10<X≤20, it represents that the low-gossypol cotton is resistant to the Aphis gossypii (resistance); when 20<X≤30, it represents that the low-gossypol cotton is of medium resistant to the Aphis gossypii (medium resistance); when 30<X≤40, it represents that the low-gossypol cotton is susceptible to the Aphis gossypii (susceptibility); and when X>40, it represents that the low-gossypol cotton is highly susceptible to the Aphis gossypii (high susceptibility).

Compared with the prior art, the present disclosure has the following beneficial technical effects.

Through indoor planting, insect inoculating and rearing, and calculating of Aphis gossypii mortality rate and average Aphis gossypii productivity, the influence by the gene-edited low-gossypol cotton on Aphis gossypii is systematically and accurately evaluated.

In the present disclosure, the method has the advantages such as simple operation, low dependence and low requirements on operators during the evaluation, and avoids the defects of conventional indoor biological testing methods that require high experience of operators.

In the present disclosure, the method strictly controls the culture conditions, thus avoiding the problem that the field test takes a long time and only one batch can be conducted in a year. At the same time, the method avoids the interference by factors such as uneven occurrence of natural insect sources in the field, unstable occurrence, and natural control by natural enemies with the evaluation method and results of Aphis gossypii influenced by cotton, ensuring the repeatability and reliability of the test.

In the present disclosure, the method can be conducted under indoor controllable conditions, and the growth, development, and mortality of Aphis gossypii are used to conduct comprehensive influence evaluation. The method is also different from the field evaluation method that determines the damage of cotton plants by aphids, and provides a scientific basis and technical support for cotton breeding.

### BRIEF DESCRIPTION OF THE DRAWINGS

The present disclosure will be further described below with reference to the accompanying drawings.

The FIGURE shows a flow chart of the method for evaluating an influence by gene-edited low-gossypol cotton on Aphis gossypii in the present disclosure.

### DETAILED DESCRIPTION OF THE EMBODIMENTS

The present disclosure provides a method for evaluating an influence by gene-edited low-gossypol cotton on Aphis gossypii, including the following steps:
S1, planting the gene-edited low-gossypol cotton indoors;
S2, collecting a fully expanded young leaf from a top part of a seedling of the cotton and placing the young leaf in a medium contained in a container, and then inoculating nymphal Aphis gossypii on the young leaf;
S3, rearing the nymphal Aphis gossypii until the 6th day after it is developed into an adult Aphis gossypii and lay new nymphal Aphis gossypii, recording a number of dead adult Aphis gossypii and a number of newly laid nymphal Aphis gossypii every 24 h, and terminating investigation when all Aphis gossypii individuals die in an initial test; and
S4, calculating an Aphis gossypii mortality rate and an average Aphis gossypii productivity, and evaluating the influence of the gene-edited low-gossypol cotton on the Aphis gossypii based on the Aphis gossypii mortality rate and the average Aphis gossypii productivity.

In an example, the young leaf is collected from the cotton seedlings with consistent growth after a cotton seedling has 2 to 4 fully expanded leaves.

In an example, the medium is an agar-containing medium.

In an example, the medium has an agar concentration of 5 wt % % to 10 wt ‰.

In an example, 5 to 8 wingless Aphis gossypii individuals are inoculated on the young leaf of the cotton in each container, and the adult Aphis gossypii is removed while only 5 initially hatched nymphal Aphis gossypii individuals are retained as Aphis gossypii for the initial test after 24 h.

In an example, the rearing is conducted at 24° C.±1° C. under a humidity of 50%±10%.

In an example, the rearing has a photoperiod of L:D=16 h:8 h.

In an example, a petiole is kept intact and then inserted into the medium while the young leaves are kept completely placed on the agar-containing medium during the culture.

In an example, a fresh fully expanded young leaf from the top part of the cotton seedling is replaced every 2 d to 3 d during the rearing to ensure an adequate nutritional intake of the Aphis gossypii individuals in the test.

In an example, newly hatched nymphal Aphis gossypii individuals are removed every day during the rearing.

In an example, a calculation formula of the Aphis gossypii mortality rate is as follows:

M=n/N×100

In the formula, M represents the Aphis gossypii mortality rate, in percentage (%); n represents a number of dead aphids on the 6th day after the initial test is started; N represents a number of aphids inoculated.

In an example, a calculation formula of the average Aphis gossypii productivity is as follows:

$$X = \frac{{\sum }_{i = 1}^{10} {X}_{i}}{50}$$

In the formula, X represents the average Aphis gossypii productivity; i represents a total number of containers; Xᵢ represents a total number of nymphal Aphis gossypii produced by initial Aphis gossypii in an i-th container; and 50 represents a total number of the initial Aphis gossypii in 10 containers.

When the average Aphis gossypii productivity X≤10, it represents high resistance to the Aphis gossypii; the average Aphis gossypii productivity 10<X≤20 represents resistance to the Aphis gossypii; the average Aphis gossypii productivity 20<X≤30 represents medium resistance to the Aphis gossypii; the average Aphis gossypii productivity 30<X≤40 represents susceptibility to the Aphis gossypii; and the average Aphis gossypii productivity X>40 represents high susceptibility to the Aphis gossypii.

The technical solutions provided by the present disclosure will be further described below with reference to the accompanying examples.

### Example 1

A method for evaluating an influence by gene-edited low-gossypol cotton on Aphis gossypii included the following steps:
S1, gene-edited low-gossypol cotton PGF was planted indoors, and non-transgenic cotton variety Jin668 was used as a control; cotton seedlings with consistent growth were selected after the cotton seedlings had 2 to 4 fully expanded leaves, and fully expanded young leaves at the top of the cotton seedlings were collected;
S2, the young leaves were placed in a culture dish containing a medium containing 10 wt ‰ agar, and then inoculated with insects on the leaves, where 8 wingless Aphis gossypii individuals were inoculated into each culture dish; after 24 h, the adult Aphis gossypii were removed and only 5 initially hatched nymphal Aphis gossypii individuals were retained as the Aphis gossypii for an initial test; during the rearing, petioles were kept intact and inserted into the medium, and the young leaves were kept completely flat on the agar-containing medium; fresh cotton leaves of a same specification were replaced every 2 d to ensure that the test insects had sufficient nutritional intake; and during the rearing, newly hatched nymphal Aphis gossypii was removed every day;
S3, rearing was conducted at 24° C.±1° C., under humidity 50%±10%, with a photoperiod L:D=16 h:8 h, the nymphal Aphis gossypii until the 6th day after it was developed into an adult Aphis gossypii and laid new nymphal Aphis gossypii, a number of dead adult Aphis gossypii and a number of newly laid nymphal Aphis gossypii were recorded every 24 h, and investigation was terminated when all Aphis gossypii individuals died in an initial test; and
S4, an Aphis gossypii mortality rate and an average Aphis gossypii productivity were calculated, and the influence by the gene-edited low-gossypol cotton on the Aphis gossypii was evaluated based on the Aphis gossypii mortality rate and the average Aphis gossypii productivity.

After calculation, an average Aphis gossypii mortality rate in this example on the 6th day was 94.72%, an average Aphis gossypii productivity was 26.22, and the influence evaluation result showed medium resistance; an average Aphis gossypii mortality rate in the control group on the 6th day was 93.09%, an average Aphis gossypii productivity was 28.75, and the influence evaluation result showed medium resistance.

The results indicated that there was no significant statistical difference between the experimental results of gene-edited low-gossypol cotton PGF and non-transgenic cotton variety Jin668. This suggested that while gene-edited low-gossypol cotton reduced the content of gossypol, its performance in the ecosystem during planting was basically similar to that of traditional cotton, and did not significantly change the influence of cotton on Aphis gossypii. Moreover, since gene-edited low-gossypol cotton was similar to non-transgenic cotton in terms of insect resistance, existing integrated prevention and control strategies were still applicable, providing more options for planting management.

Specific examples are used herein to explain the principles and implementations of the present disclosure. The foregoing description of the embodiments is merely intended to help understand the method of the present disclosure and its core ideas; besides, various modifications may be made by a person of ordinary skill in the art to specific embodiments and the scope of application in accordance with the ideas of the present disclosure. In conclusion, the content of the description shall not be construed as limitations to the present disclosure.

What is claimed is:

1. A method for evaluating an influence by a gene-edited low-gossypol cotton on Aphis gossypii, comprising the following steps:
S1, planting the gene-edited low-gossypol cotton indoors;
S2, collecting a fully expanded young leaf from a top part of a seedling of the gene-edited low-gossypol cotton and placing the young leaf in a medium in a container, and then inoculating nymphal Aphis gossypii on the young leaf;
S3, rearing the nymphal Aphis gossypii until the 6th day after it is developed into an adult Aphis gossypii and lay new nymphal Aphis gossypii, recording a number of dead adult Aphis gossypii and a number of newly laid nymphal Aphis gossypii every 24 h, and terminating the recording when all Aphis gossypii individuals die in an initial test; and
S4, calculating an Aphis gossypii mortality rate and an average Aphis gossypii productivity, and evaluating the influence by the gene-edited low-gossypol cotton on the Aphis gossypii based on the Aphis gossypii mortality rate and the average Aphis gossypii productivity.

2. The method for evaluating an influence by the gene-edited low-gossypol cotton on Aphis gossypii according to claim 1, wherein the young leaf is collected from the seedling of the gene-edited low-gossypol cotton with consistent growth after the seedling of the gene-edited low-gossypol cotton has 2 to 4 fully expanded leaves.

3. The method for evaluating an influence by the gene-edited low-gossypol cotton on Aphis gossypii according to claim 1, wherein the medium is an agar-containing medium.

4. The method for evaluating an influence by the gene-edited low-gossypol cotton on Aphis gossypii according to claim 3, wherein the medium has an agar concentration of 5 wt ‰ to 10 wt ‰.

5. The method for evaluating an influence by the gene-edited low-gossypol cotton on Aphis gossypii according to claim 1, wherein 5 to 8 wingless Aphis gossypii individuals are inoculated on the young leaf of the cotton in each container, and the adult Aphis gossypii is removed while only 5 initially hatched nymphal Aphis gossypii individuals are retained as Aphis gossypii of the initial test after 24 h.

6. The method for evaluating an influence by the gene-edited low-gossypol cotton on Aphis gossypii according to claim 1, wherein the rearing is conducted at 24° C.±1° C. under a humidity of 50%±10%.

7. The method for evaluating an influence by the gene-edited low-gossypol cotton on Aphis gossypii according to claim 1, wherein the culture has a photoperiod of L:D=16:8.

8. The method for evaluating an influence by the gene-edited low-gossypol cotton on Aphis gossypii according to claim 1, wherein a fresh fully expanded young leaf from the top part of the cotton seedling is replaced every 2 d to 3 d during the rearing to ensure an adequate nutritional intake of the Aphis gossypii individuals in the initial test.

9. The method for evaluating an influence by the gene-edited low-gossypol cotton on Aphis gossypii according to claim 1, wherein newly hatched nymphal Aphis gossypii individuals are removed every day during the culture.

10. The method for evaluating an influence by the gene-edited low-gossypol cotton on Aphis gossypii according to claim 1, wherein the influence is evaluated as follows:
$$X = \frac{{\sum }_{i = 1}^{10} {X}_{i}}{50} ,$$
wherein X represents an average Aphis gossypii productivity; i represents a total number of containers; Xᵢ represents a total number of nymphal Aphis gossypii produced by initial Aphis gossypii in an i-th container; and 50 represents a total number of the initial Aphis gossypii in 10 containers; and
when X≤10, it represents a high resistance of the low-gossypol cotton to the Aphis gossypii; when 10<X≤20, it represents a resistance of the low-gossypol cotton to the Aphis gossypii; when 20<X≤30, it represents a medium resistance of the low-gossypol cotton to the Aphis gossypii; when 30<X≤40, it represents a susceptibility of the low-gossypol cotton to the Aphis gossypii; and when X>40, it represents a high susceptibility of the low-gossypol cotton to the Aphis gossypii.
